(19) United States
(12) Patent Application Publication
(10) Pub. No.: US 2017/0209157 A1
(43) Pub. Date: Jul. 27, 2017

(54) DEVICE AND METHODS FOR OSTEOPERFORATION

(71) Applicant: PROPEL ORTHODONTICS, LLC, Ossining, NY (US)

(72) Inventors: Bryce A. WAY, San Jose, CA (US); Richard JOHNSON, Briarcliff Manor, NY (US); Peter MIGNEAULT, Succasunna, NJ (US); Gerald ZILLES, Phoenix, AZ (US); Christopher U. PHAN, San Leandro, CA (US); Phillip ABATELLI, Wesbury, NY (US); Maria V. NUESTRO, San Jose, CA (US); Amin MIRZAAGHAEIAN, Fremont, CA (US)

(21) Appl. No.: 15/329,586

(22) PCT Filed: Jul. 29, 2015
(86) PCT No.: PCT/US2015/042756

§ 371 Date: Jan. 27, 2017

Related U.S. Application Data
(60) Provisional application No. 62/030,536, filed on Jul. 29, 2014.

(51) Int. Cl. A61B 17/16; A61C 7/02; A61C 7/12

(52) U.S. Cl. CPC A61B 17/1673; A61C 7/12; A61C 7/02

(57) ABSTRACT

A device for increasing movement of a tooth in a jaw includes a handle, an elongate member extending from the handle, and a screw tip at a distal end of the elongate member. The screw tip is configured to drill into cortical bone of the jaw to increase movement of the tooth. Further, the screw tip is configured to be removable from the handle.

### CROSS-REFERENCE TO RELATED APPLICATIONS

This application claims priority to U.S. Provisional Application No. 62/030,536, filed Jul. 29, 2014, the entirety of which is incorporated by reference herein.

This application may be related to patent application Ser. No. 13/471,099, titled “METHOD AND DEVICE FOR CAUSING TOOTH MOVEMENT,” now U.S. Pat. No. 8,602,777, the entire contents of which are incorporated by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure relates to dental and orthodontic devices and methods, particularly devices and methods for increasing the movement of teeth in the jaw.

### BACKGROUND

A large percentage of today's children and adult population undergo orthodontic treatments at some point in their lives to treat malocclusions (i.e., crooked teeth leading to poor bite) or improve skeletal abnormalities. Because growth and development of adult teeth is generally stagnant, treatment of malocclusions in adults requires reliance on the dento-alveolar element, e.g. the ability of teeth to move when a sufficient inflammatory response is created in the jaw.

The most common method of creating movement in teeth is through the use of braces. The braces include wires and other tensioning devices, such as rubber bands and coils or removable trays, that exert a continuous force on the tooth to move the tooth to a desired location. The use of braces to cause tooth movement, however, takes on average 18-24 months and can take up to 3-4 years, often causing both social and physical discomfort.

Accordingly, it would be advantageous to have a treatment method that could successfully move a tooth or teeth in a shorter period of time.

### SUMMARY

In general, in one embodiment, a device for forming holes in the cortical bone adjacent a tooth in order to increase movement of the tooth includes a handle, an elongate member extending from the handle, a screw tip at a distal end of the elongate member, and a cap. The screw tip is configured to drill into cortical bone to increase movement of the tooth.

The cap includes an elongate portion configured to cover the screw tip, an attachment mechanism configured to attach the elongate member, and a distal portion having a flat distal surface that is substantially perpendicular to a longitudinal axis of the screw tip. The flat distal surface has a wider diameter than a diameter of the elongate member.

This and other embodiments can include one or more of the following features.

The cap can further include angled supports extending from the elongate portion to the distal portion. The attachment mechanism can be a snapping mechanism. The elongate member can include a ridge thereon. The snapping mechanism can be configured to snap over the ridge. The device can further include a spring biased sleeve. The sleeve can be configured to rest against tissue during use and allow movement of the elongate member with respect to the sleeve to vary the length of exposed screw tip as force is applied to the handle. The handle can include a first end attached to the elongate member and a second end. The first end can be rotatable with respect to the second end. The first end can be configured to control rotation of the screw tip.

In general, in one embodiment, a device for forming holes in the cortical bone adjacent a tooth in order to increase movement of the tooth includes a handle, an elongate member extending from the handle, a screw tip at a distal end of the elongate member, and a release mechanism on a proximal end of the handle. The screw tip is configured to drill into cortical bone to increase movement of the tooth. The release mechanism is configured to allow the release of the screw tip from the handle when activated by a user.

This and other embodiments can include one or more of the following features. The release mechanism can include a button. The button can be set distally into a proximal end of the handle. The device can further include a ball and detent system configured to interact with the release mechanism to allow the release or locking of the screw tip from the handle. The elongate member can further include a plurality of longitudinally extending ribs configured to interact with a portion of the handle to rotationally lock the elongate member relative to the handle. The elongate member can include an annular slot and the handle can include a bearing configured to interact with the annular slot to hold the elongate member in the handle. The device can further include a spring biased sleeve. The sleeve can be configured to rest against tissue during use and allow movement of the elongate member with respect to the sleeve to vary the length of exposed screw tip as force is applied to the handle. The handle can include a first end attached to the elongate member and a second end, and the first end can be rotatable with respect to the second end. The first end can be configured to control rotation of the screw tip.

In general, in one embodiment, a device for forming holes in the cortical bone adjacent a tooth in order to increase movement of the tooth includes a handle, an elongate member extending from the handle, and a screw tip at a distal end of the elongate member. The screw tip is configured to drill into cortical bone to increase movement of the tooth. The screw tip has an asymmetric distal-most point.

This and other embodiments can include one or more of the following features. A pitch of a thread of the screw tip can be between 0.025 inches and 0.050 inches. A thread of the screw tip can extend approximately 0.35 to 0.50 inches in length. The device can further include a spring biased sleeve. The sleeve can be configured to rest against tissue during use and allow movement of the elongate member with respect to the sleeve to vary the length of exposed screw tip as force is applied to the handle. The handle can include a first end attached to the elongate member and a second end, and the first end can be rotatable with respect to the second end. The first end can be configured to control rotation of the screw tip.

In general, in one embodiment, a method of increasing movement of a tooth in a jaw includes: (1) holding a handle of a device with a single hand, the device having an elongate member extending from the handle and a screw tip at a distal end of the elongate member; (2) drilling a hole with the screw tip through a cortical bone of a jaw by rotating a distal portion of the handle with the single hand and holding a proximal portion stationary, wherein the jaw comprises at least one tooth having an orthodontic brace thereon, the drilling performed to increase rate of movement of the tooth; and (3) after drilling, releasing the elongate member from the handle by activating a release mechanism.

This and other embodiments can include one or more of the following features. The release mechanism can be a button on a proximal end of the handle. Drilling a hole can include drilling a hole in a mesial surface of the jaw. The method can further include drilling a plurality of holes spaced along a mesial surface of the jaw. Drilling a hole can include drilling a hole in a gingival flap. Drilling a hole can include drilling the hole proximal to a central or lateral tooth or drilling a hole in the palatal.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an osteoperforation device as described herein with the tip covered by a sleeve.

FIG. 1B shows a distal tip for a device similar to that shown in FIG. 1A with the tip covered by a sleeve.

FIG. 1C shows a distal tip with a cap thereon for a device similar that shown in FIG. 1A with a distal end cap.

FIG. 1D shows a side view of the device of FIG. 1A with the tip exposed.

FIG. 1E shows a cross section of the device of FIG. 1A with the tip exposed.

FIGS. 2A-2C show various views of an elongate member and distal tip for an osteoperforation device similar to that shown in FIG. 1A. FIG. 2A is a cross-section. FIG. 2B is a perspective view. FIG. 2C is a side view.

FIGS. 3A-3C show a handle for an osteoperforation device similar to that shown in FIG. 1A. FIG. 3A is a perspective view. FIG. 3B is an exploded view. FIG. 3C is a cross-section.

FIGS. 4A-4B show the cross-section of a distal tip of an osteoperforation device similar to that shown in FIG. 1A with a distal end cap. FIG. 4A shows a sleeve fully covering the distal tip. FIG. 4B shows an exposed distal tip.

FIGS. 5A-5C show various views of a screw tip for an osteoperforation device.

FIGS. 6A-6B show use of an exemplary osteoperforation device.

FIGS. 7A-7C show a ball detent system for attaching and detaching a shaft of an osteoperforation device from a handle. FIG. 7A is a cross-section. FIG. 7B is a side view. FIG. 7C is an exploded perspective view.

FIGS. 8A-8C show a proximal end of a handle. FIG. 8A is a side view. FIG. 8B is a perspective view. FIG. 8C is a cross-section.

FIGS. 9A and 9B show a stopper configured to lock the screw tip in an exposed position. FIG. 9A is a perspective view. FIG. 9B is a cross-section.

### DETAILED DESCRIPTION

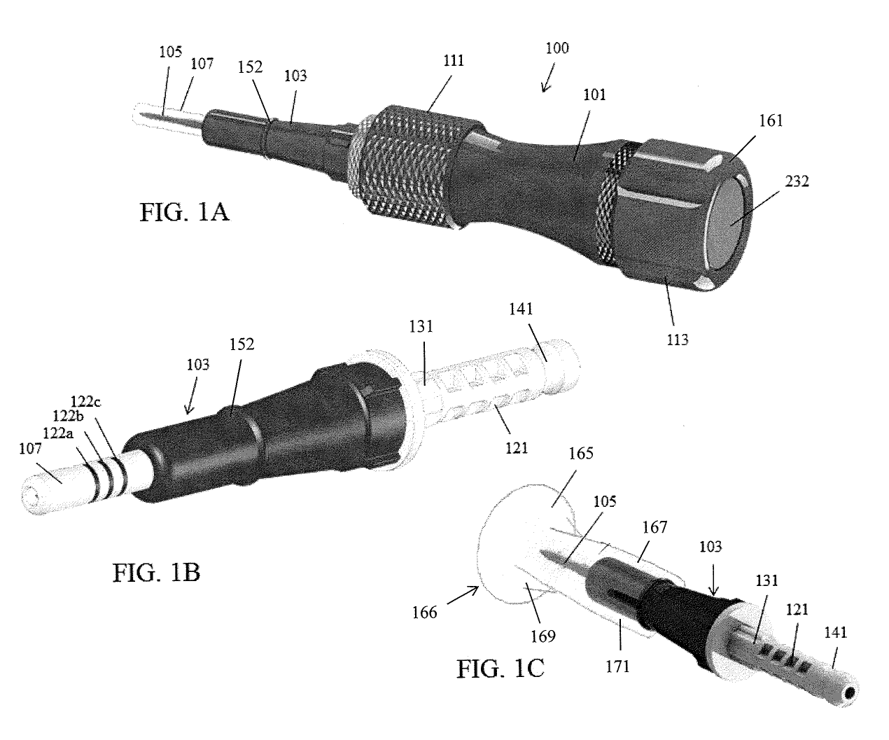

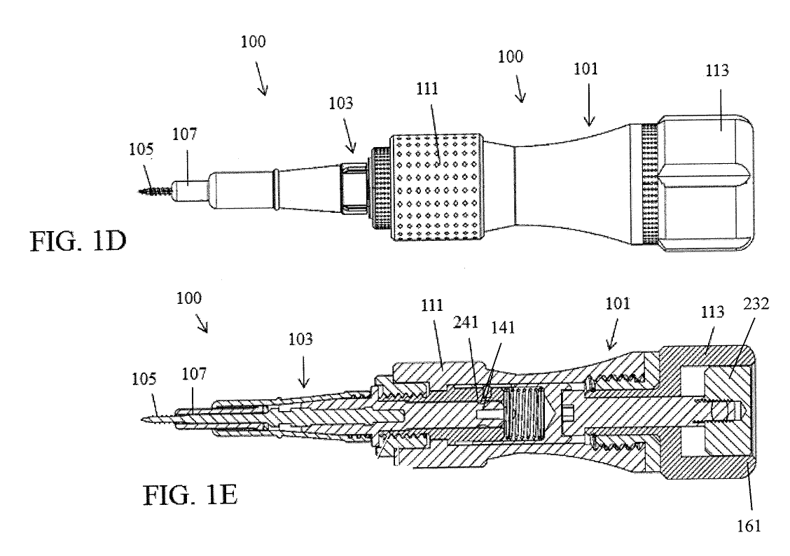

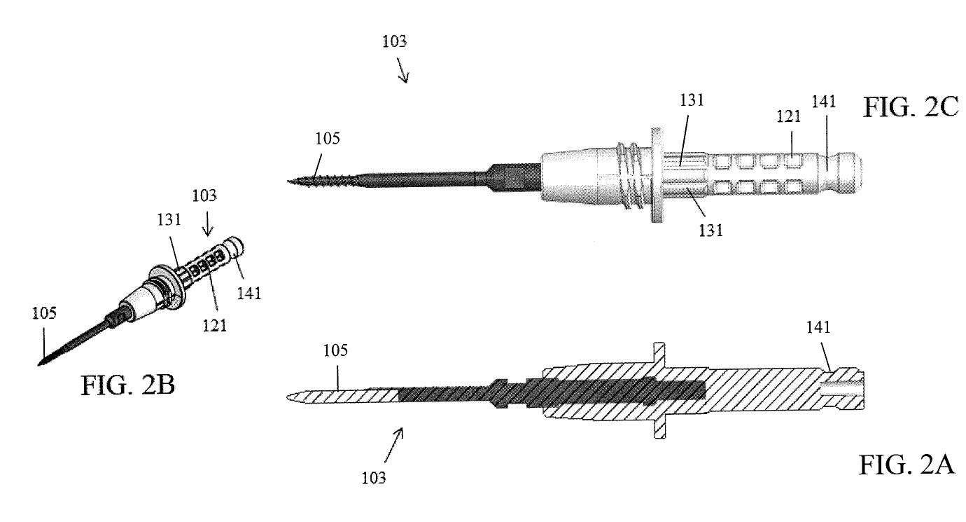

Referring to FIGS. 1A-4B, a device 100 is a hand-held instrument to create micro-osteoperforation in bone and soft tissue. The device 100 can be used, for example, to increase the movement of a tooth in a jaw. The device 100 includes a handle 101 and an elongate separable shaft 103 extending from the handle 101. A screw tip 105, for example made of stainless steel, can be located at the distal end of the elongate shaft 103.

The shaft 103 and screw tip 105 can be rotatable with respect to the handle 101 or a portion of the handle 101. For example, the handle 101 can include a first end 111 attachable to the shaft 103 and a second end 113 configured to be held stationary by the user. The first end 111 can be rotatable with respect to the second end 113 so as to control rotation of the shaft 103, and hence rotation of the screw tip 105.

In some embodiments, a sleeve 107 can be configured to move along the screw tip 105 to vary the length of exposed screw tip 105. The screw length can thus vary from 0 mm to 7 mm. The sleeve 107 can be pushed into the elongate shaft 103 to set the length of exposed screw tip 105. In some embodiments, the sleeve 107 can include markers 122 thereon configured to identify the amount of screw tip exposed as the sleeve 107 is moved into the elongate shaft 103. For example, when the markers 122 align with a distal edge of the elongate shaft 103, it can indicate the length of the exposed screw tip, such as at 3 mm, 5 mm, and 7 mm. Further, in some embodiments, a stop 154 can prevent the sleeve 107 from moving past a particular point, such as to expose the tip more than 7 mm.

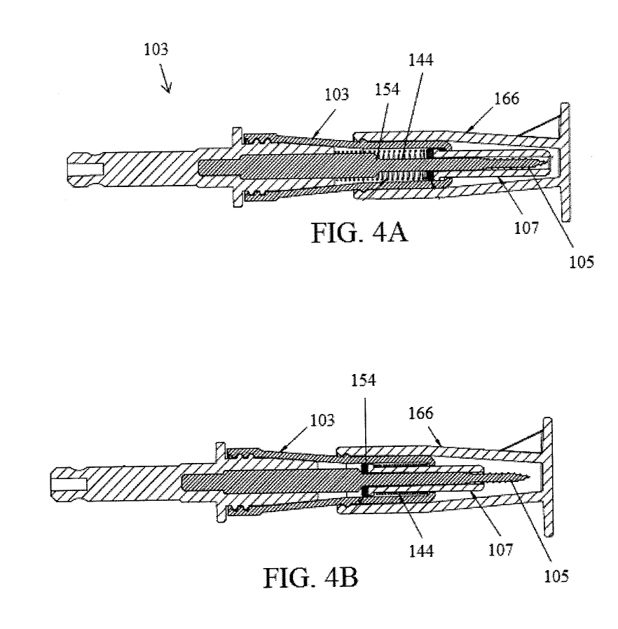

In some embodiments, a spring 144 (see FIGS. 4A and 4B) can be used to bias the sleeve 107 over the distal tip 105. Thus, as the screw tip 105 is drilled into the gums, proximal pressure can be placed on a distal end of the sleeve 107, pushing the spring 144 proximally and exposing more of the screw tip 105. In other embodiments, the sleeve 107 can be biased into the elongate body 103 so that the screw tip 105 is always exposed.

In some embodiments, the sleeve 107 can be configured to remain stationary as the elongate body 103 and screw tip 105 rotate. This can advantageously ensure that the sleeve 105 won't spin against the gums as the screw tip 105 is drilled into the mouth, thereby protecting the surface of the gums from irritation or abrasion.

As shown in FIGS. 1C and 4A-4B, a protective distal cap 166 can be used to cover the distal end of the device 100 when not in use. Referring to FIG. 1C, the cap 166 can include an elongate portion 167 that covers at least the screw tip 105 as well as a distal end 165 that is configured to allow the device 100 to sit or rest on its distal end. The distal end 165 can thus include a flat distal surface, such as a circular surface, that is perpendicular to a central longitudinal axis of the device and has a larger diameter than the rest of the cap 166 and/or than the elongate body 103. Further, struts 169, such as triangular struts, can connect the elongate portion 167 and the distal end 165 to provide stabilizing support when the device is inserted into the cap 166 and rested thereon. Further, a proximal end of the cap 166 can include snapping features 171, such as collets or living hinges, that can snap over a portion of the device 100, such as over a rib 152 (see FIGS. 1A-1B) located between the rotatable first end 111 and the screw tip 105. The cap 166 can not only allow resting of the device 100 on its distal end, but can also prevent contamination of the tip and/or protect users or medical personnel from being cut by the tip when not in use.

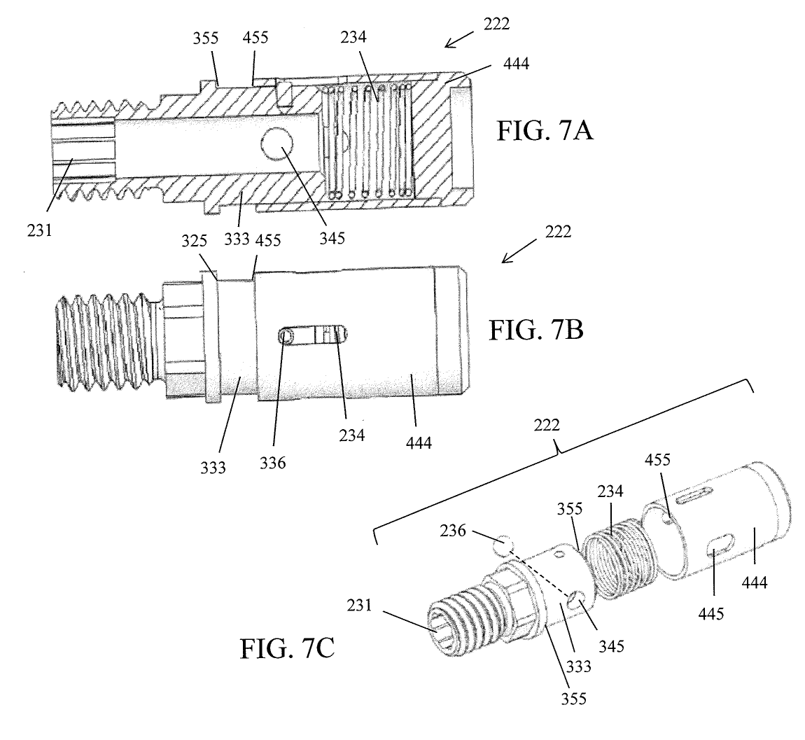

In some embodiments, the shaft 103 can be separable from the handle 101. The shaft 103 can include an attachment portion that is configured to snap or screw into the handle 101. For example, the shaft 103 can include teeth 121 that mate with teeth on the inside of the handle 101. Likewise, the shaft 103 can include a star locking mechanism 131 that mates with a corresponding star locking mechanism 231 (see FIGS. 7C) on the inside of the handle. Finally, the shaft 103 can include a proximal annular slot 141 configured to engage with one or more bearings 236 on the inside of the handle 101. These various features, alone or in combination, can serve to hold the sleeve within the handle 101 with a bib-style lock.

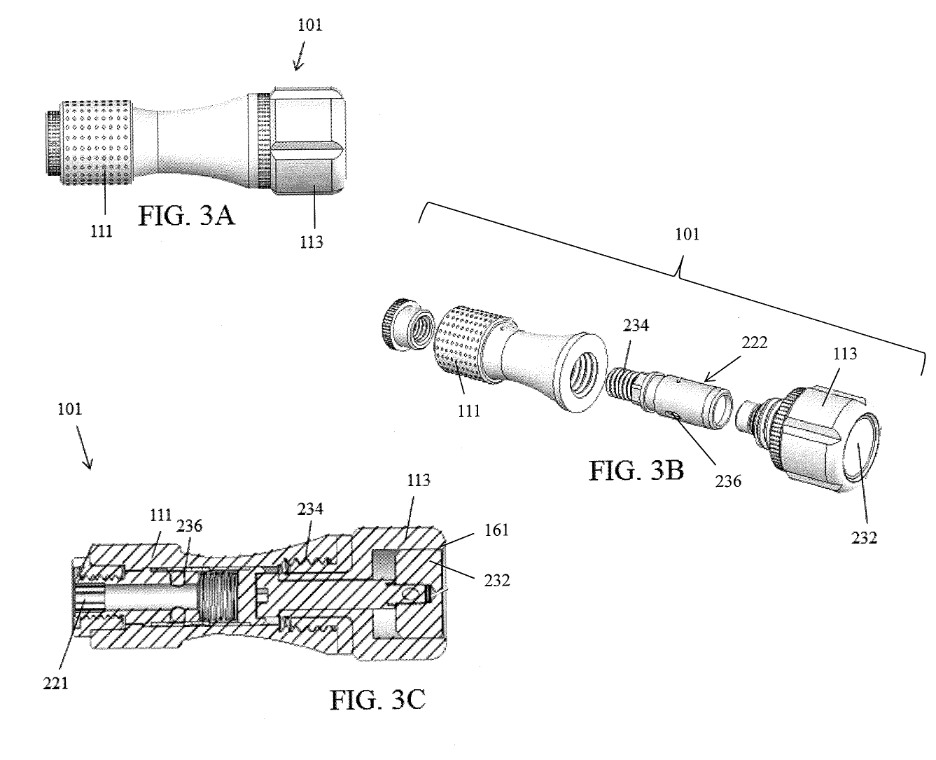

Referring to FIG. 3B, a button 232 on the handle 101 can be used to manually release the elongate member 103 from the locked position through a ball detent system 222. The button 232 can be set in distally from the proximal-most edge 161 of the handle 101.

The ball detent system is shown more clearly in FIGS. 7A-7C. The ball-detent system 222 includes a distal portion 333, a proximal portion 444, and a spring 234 therebetween. A first bearing hole 345 in the distal portion 333 can be configured to house a bearing 236 that engages with the proximal slot 141 (see FIGS. 1B and 1C) to hold the shaft 103 in place in the handle 101. Further, the proximal portion 444 can have a second bearing hole 445. When the button 232 is pushed, compression on the spring 234 can allow the proximal portion 444 to move over the distal portion 333 until a distal ledge or rim 355 on the distal portion 333 comes into contact with a distal edge 455 of the proximal portion 444 and the holes 345, 445 overlap. In this position, the bearing 236 can pop into the ball slot 445, thereby releasing the bearing 236 from the slot 141 in the shaft 103. Likewise, to place the shaft 103 back into the handle 101, the button 232 can be pushed again to move the bearing 236 into the ball slot 445 and out of the way of the shaft 103. Once the shaft 103 is pushed all the way in, the button can be released, and the bearing 236 can be locked into the slot 141.

The locking mechanism can advantageously be releasable (e.g., the bearings 236 can snap out of the slot 141) if a high enough proximally directed axial force is placed on the handle, ensuring that the screw tip 105 will not be pulled out of a patient's jaw unintentionally. Further, the release mechanism can advantageously occur automatically when the button 232 is pushed. Accordingly, if held, for example, over a hazardous waste container, the user can push the button 232 and the elongate member 103 can automatically drop into the container. Advantageously, by having the shaft 103 be removable from the handle 101, the handle 101 can be used with different shafts 103, thereby allowing the shafts 103 to be disposable and the handle 101 to be reusable. In some embodiments, the reusable handle can be configured to work with various interchangeable tips, such as both with a tip with a retractable sleeve and with a tip that is fully exposed.

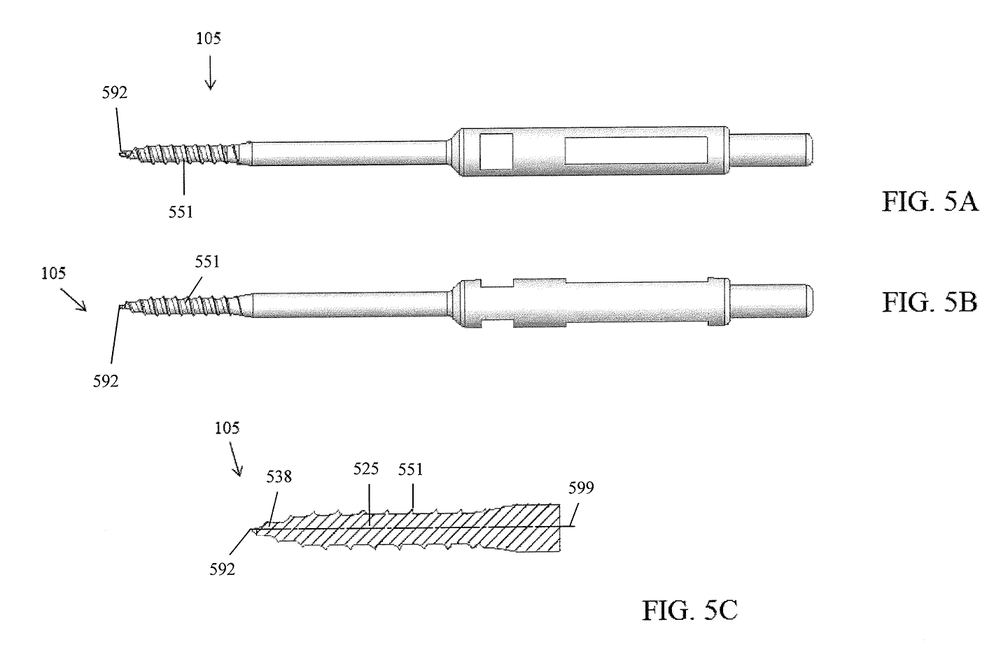

Referring to FIGS. 5A-5C, the screw tip 105 can be shaped so as to easily drive into the gums. The thread or ribbon 551 on the screw can begin at the distal-most point 592 and extend off-center relative to the central axis 599 from the point 592 down the length of the tip 105 (see FIG. 5C). Thus, rather than having a symmetrical point on the screw, the sharp point 592 can be asymmetric, formed by the spiraling ribbon 551. Further, the tip 105 can include a cylindrical portion 525 covered in thread and then an angled portion 535 (e.g., angled at 10-20 degrees relative to the central axis 599, such as approximately 13 degrees) also covered in the threads of the screw, which then extends to a point formed by just the ribbon 551.

In one embodiment, the thread can extend a length of approximately 0.35 to 0.50 inches, such as approximately 0.38 inches. Further, the pitch of the screw threads can be between 0.025 and 0.050 inches, such as between 0.030 and 0.045 inches, such as approximately 0.039 inches. The pitch and asymmetric tip shape can advantageously allow the tip to screw into the gums with minimal force.

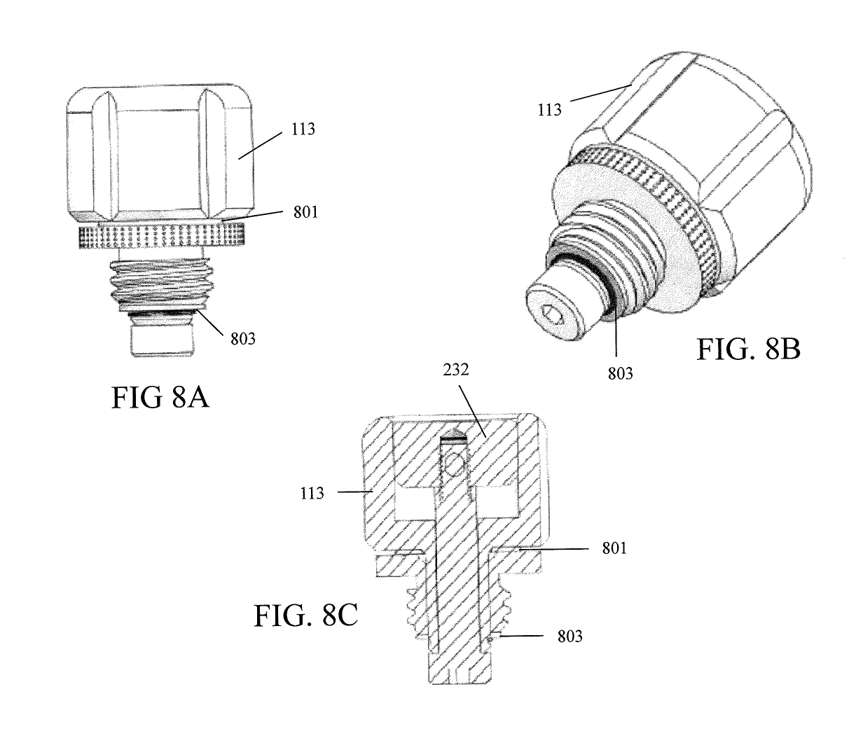

Referring to FIGS. 8A-8C, in some embodiments, washers 801, 803 can be used to provide a smooth surface between one or more parts of the handle 101. The washers 801, 803 can be, for example, made of Teflon. Washers 801, 803 can reduce friction to both make rotation easier and reduce noise associated with rotation of portions of the device.

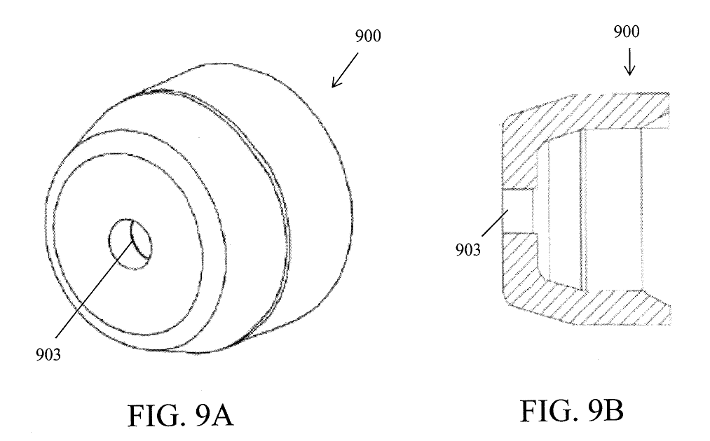

Further, referring to FIGS. 9A-9B, in some embodiments, a stopper 900 can be used to hold the sleeve in a proximal position to set the length of exposed tip. That is, the stopper 900 can include a hole 903 therethrough that can slide over the screw tip and the sleeve. The stopper can be made, for example, of a polymer, such as polycarbonate, that can stretch around the sleeve for placement and then hold the sleeve tightly in place.

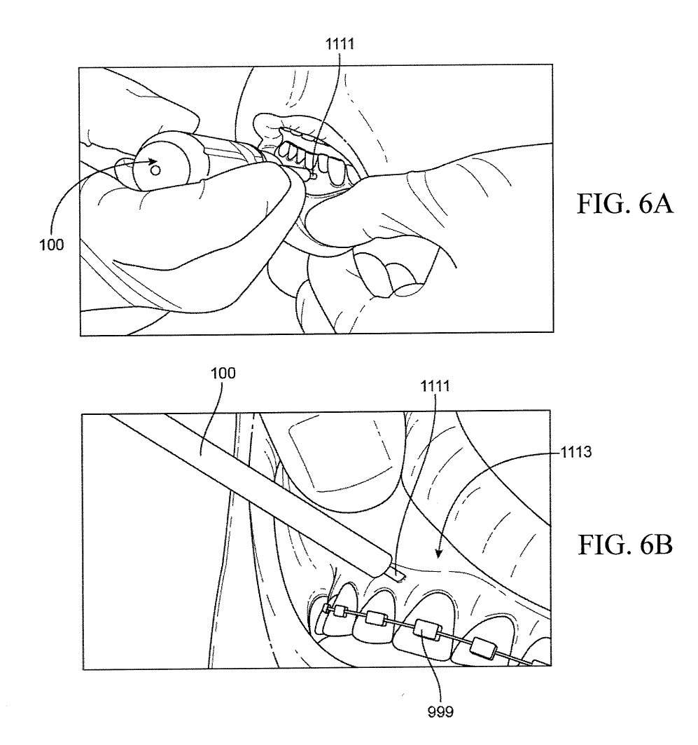

The device 100 can be used to enhance the movement of a tooth or teeth in a jaw. For example, referring to FIGS. 6A and 6B, the device 100 can be used to form perforations or holes 1111 in the jaw of a patient, called “osteoperforation.” To do so, an elongated member 103 (such as a disposable elongated member 103) can be placed into a handle 101 (such as a reusable handle). The device 100 can be held at approximately a 90 degree angle to the patient's gingival while keeping the tissue taunt. The screw tip 105 can be rotated against the gums by rotating the first part 111 of the handle 101, for example in a clockwise direction. Pressure can be applied to the device 100, which, in combination with the rotation of the screw tip 105, can cause a cutting edge of the screw tip to form one or more holes, such as between 1 and 10 holes, for example approximately 3 holes, in the gingival flap of the jaw 113, for example through a mesial surface of the jaw and/or through cortical bone of the jaw.

Each hole in the jaw can be formed without cutting away a gingival flap prior to formation of the hole. Further, each hole can be formed in the cortical bone near a malocclusion sought to be treated. The pressure and rotation can be stopped when the desired depth has been reached, i.e. when the screw tip 105 has been advanced all the way into the jaw and further penetration has been stopped by the sleeve 107. Holes of between 0 mm and 10 mm deep can be formed, such as holes of approximately 3 mm, 5 mm, or 7 mm. The handle 101 can be rotated in the opposite direction, for example counter-clockwise, to remove the device from the jaw.

The button 232 can then be pushed to release the elongate member 103 from the handle 101.

The device 100 can advantageously be held and rotated with a single hand (e.g., the user can hold the second end 113 stationary in the palm of the hand while rotating the first end 111 with the tips of the thumb and index finger). Further, when the proximal-most edge 161 of the second end 113 is set against the palm, the inset button 232 can be protected or covered by palm, thereby preventing accidental release of the tip 105 while the device 100 is in use.

Referring to FIG. 6B, in some embodiments, the devices described herein, such as device 100, can be used in conjunction with braces 999 or other orthodontic devices. In other embodiments, as shown in FIG. 6A, the devices described herein can be used on teeth do not have braces (such as after an injury to help move the tooth back in place).

The holes 1111 formed in the jaw can create an inflammatory response within the jaw. As a result, osteoclast precursors and cytokines can be drawn to the site of the holes 1111. The cytokines can promote osteoclast formation and activation, causing increased bone remodeling and movement. The holes 1111 formed in the jaw can thus allow a tooth or teeth to move over time to partially or fully treat the malocclusion.

The devices described herein can be used to correct major molar uprighting, major lower molar protraction, major canine protraction, and major intrusion, as described in U.S. patent application Ser. No. 13/471,099, titled “METHOD AND DEVICE FOR CAUSING TOOTH MOVEMENT,” now U.S. Pat. No. 8,602,777, the entire contents of which are incorporated by reference.

Use of the device described herein for osteoperforation advantageously taps a bone metabolism process that safely accelerates motion. The microperforation process using the devices described herein is safe, simple, and produces local alveolar bone reactions that enable rapid motion of teeth. Further, the process can be performed in-office and, as described above, can be performed precisely for a broad range of patients and in a broad range of different types of teeth.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the examples described herein, but only by the plain meaning of the claim terms employed.

What is claimed is:

1. A device for forming holes in the cortical bone adjacent a tooth in order to increase movement of the tooth, the device comprising:
a handle;
an elongate member extending from the handle;
a screw tip at a distal end of the elongate member, wherein the screw tip is configured to drill into cortical bone to increase movement of the tooth; and
a cap, the cap including:
an elongate portion configured to cover the screw tip;
an attachment mechanism configured to attach the elongate member; and
a distal portion having a flat distal surface that is substantially perpendicular to a longitudinal axis of the screw tip, the flat distal surface having a wider diameter than a diameter of the elongate member.

2. The device of claim 1, wherein the cap further includes angled supports extending from the elongate portion to the distal portion.

3. The device of claim 1, wherein the attachment mechanism is a snapping mechanism.

4. The device of claim 1, wherein the elongate member includes a ridge thereon, the snapping mechanism configured to snap over the ridge.

5. The device of claim 1, further comprising a spring biased sleeve, the sleeve configured to rest against tissue during use and allow movement of the elongate member with respect to the sleeve to vary the length of exposed screw tip as force is applied to the handle.

6. The device of claim 1, wherein the handle comprises a first end attached to the elongate member and a second end, the first end rotatable with respect to the second end.

7. The device of claim 6, wherein the first end is configured to control rotation of the screw tip.

8. A device for forming holes in the cortical bone adjacent a tooth in order to increase movement of the tooth, the device comprising:
a handle;
an elongate member extending from the handle;
a screw tip at a distal end of the elongate member, wherein the screw tip is configured to drill into cortical bone to increase movement of the tooth; and
a release mechanism on a proximal end of the handle configured to allow the release of the screw tip from the handle when activated by a user.

9. The device of claim 8, wherein the release mechanism comprises a button.

10. The device of claim 9, wherein the button is set distally into a proximal end of the handle.

11. The device of claim 8, further comprising a ball and detent system configured to interact with the release mechanism to allow the release or locking of the screw tip from the handle.

12. The device of claim 8, wherein the elongate member further includes a plurality of longitudinally extending ribs configured to interact with a portion of the handle to rotationally lock the elongate member relative to the handle.

13. The device of claim 8, wherein the elongate member includes an annular slot and the handle includes a bearing configured to interact with the annular slot to hold the elongate member in the handle.

14. The device of claim 8, further comprising a spring biased sleeve, the sleeve configured to rest against tissue during use and allow movement of the elongate member with respect to the sleeve to vary the length of exposed screw tip as force is applied to the handle.

15. The device of claim 8, wherein the handle comprises a first end attached to the elongate member and a second end, the first end rotatable with respect to the second end.

16. The device of claim 15, wherein the first end is configured to control rotation of the screw tip.

17. A device for forming holes in the cortical bone adjacent a tooth in order to increase movement of the tooth, the device comprising:
a handle;
an elongate member extending from the handle; and
a screw tip at a distal end of the elongate member, wherein the screw tip is configured to drill into cortical bone to increase a rate of movement of the tooth, the screw tip having an asymmetric distal-most point.

18. The device of claim 17, wherein a pitch of a thread of the screw tip is between 0.025 inches and 0.050 inches.

19. The device of claim 17, wherein a pitch of a thread of the screw tip extends approximately 0.35 to 0.50 inches in length.

20. The device of claim 17, further comprising a spring biased sleeve, the sleeve configured to rest against tissue during use and allow movement of the elongate member with respect to the sleeve to vary the length of exposed screw tip as force is applied to the handle.

21. The device of claim 17, wherein the handle comprises a first end attached to the elongate member and a second end, the first end rotatable with respect to the second end.

22. The device of claim 21, wherein the first end is configured to control rotation of the screw tip.

23. A method of increasing movement of a tooth in a jaw, the method comprising:
holding a handle of a device with a single hand, the device having an elongate member extending from the handle and a screw tip at a distal end of the elongate member;
drilling a hole with the screw tip through a cortical bone of a jaw by rotating a distal portion of the handle with the single hand and holding a proximal portion stationary, wherein the jaw comprises at least one tooth having an orthodontic brace thereon, the drilling performed to increase a rate of movement of the tooth; and
after drilling, releasing the elongate member from the handle by activating a release mechanism.

24. The method of claim 23, wherein the release mechanism is a button on a proximal end of the handle.

25. The method of claim 23, wherein drilling a hole comprises drilling a hole in a mesial surface of the jaw.

26. The method of claim 25, further comprising drilling a plurality of holes spaced along a mesial surface of the jaw.

27. The method of claim 23, wherein drilling a hole comprises drilling a hole in a gingival flap.
